(19) United States
(12) Patent Application Publication
(10) Pub. No.: US 2023/0329993 A1
(43) Pub. Date: Oct. 19, 2023

(54) PERSONAL CLEANSING COMPOSITION FREE OF ALKYL SULFATE OR ALKYL ETHER SULFATE TYPE OF SURFACTANTS

(71) Applicant: The Procter & Gamble Company, Cincinnati, OH (US)

(72) Inventors: Lesheng Zhang, Beijing (CN); Ye Li, Beijing (CN); Ruiqi Lu, Beijing (CN); Karl Shiqing Wei, Mason, OH (US); Edward Dewey Smith, III, Mason, OH (US)

(21) Appl. No.: 18/299,864

(22) Filed: Apr. 13, 2023

(30) Foreign Application Priority Data
Apr. 14, 2022 (WO) PCT/CN2022/086854

(51) Int. Cl. A61K 8/46; A61K 8/60; A61Q 19/10

(52) U.S. Cl. CPC A61K 8/466; A61K 8/602; A61Q 19/10; A61K 2800/33; A61K 2800/30

(57) ABSTRACT

A personal cleansing composition includes a surfactant system, where the surfactant system contains from about 0.1% to about 5% of a fatty acyl isethionate surfactant and from about 0.5% to about 40% of a co-surfactant. The composition is free of alkyl sulfate or alkyl ether sulfate type of surfactants. The composition also contains from about 0.05% to about 5% of a of a non-ionic surfactant comprising a fatty alkanolamide, from about 0.01% to about 2.0% of a hydrophobically modified ethoxylated methyl glucoside, and from about 0.01% to about 1.0% of a preservative. The pH of the composition is from about 5.5 to about 7.

### FIELD OF THE INVENTION

The present application generally relates to personal cleansing compositions with an increased stability both in terms of phase, color and microbial stability, their methods and their uses. The personal cleansing compositions comprise a surfactant system, wherein the surfactant system comprises a fatty acyl isethionate surfactant and when the personal cleansing composition is free of alkyl sulfate or alkyl ether sulfate type of surfactants.

### BACKGROUND OF THE INVENTION

Personal cleansing compositions have traditionally been marketed in a variety of forms such as bar soaps, creams, lotions, and gels. Typically, these products must satisfy a number of criteria to be acceptable to consumers. These criteria include cleansing effectiveness, skin feel, mildness to skin, hair, and ocular mucosae, and lather volume. Ideal personal cleansers should gently cleanse the skin or hair, cause little or no irritation, and should not leave the skin or hair overly dry after frequent use.

Anionic surfactants are widely used in personal cleansing compositions. Many of these anionic surfactants contain elongated micelles and are viscoelastic, which is of great importance, especially in the design of shampoos and body washes. In most personal cleansing compositions, alkyl sulfate or alkyl ether sulfate as the anionic surfactants predominate.

The formulation of environmentally friendly personal cleansing compositions is becoming a major challenge for satisfying a new expectation of consumers, in particular that of ecologically designed and/or natural products. It becomes necessary to propose personal cleansing compositions free of alkyl sulfate and alkyl ether sulfate, which have good cosmetic qualities, mainly in terms of viscosity, creamy lather, and clean skin feel.

Consumers prefer sulfate-free personal cleansing compositions due to perceived mildness and desirable sensorial experience. There is an interest to provide personal cleansing products that comprise alternative mild surfactant systems with relatively improved ecotoxic or ecologically friendly environmental profile.

Personal cleansing compositions having a surfactant system comprising a fatty acyl isethionate surfactant and being free of alkyl sulfate or alkyl ether sulfate type of surfactants have been developed. Fatty acyl isethionates are mild anionic surfactants highly desirable in personal cleansing products for hair or skin, because fatty acyl isethionates can lather well, are mild to the skin and have good emollient properties.

However, fatty acyl isethionates may result in unstable personal cleansing compositions which can exhibit inconsistent rheology profiles. It has been observed phase separation of personal cleansing compositions comprising a fatty acyl isethionate and a fragrance component at 40° C. after 3 months. The phase separation might be attributed to the presence of hydrophobic molecules in the fragrance component or fatty acid that has been generated from the hydrolysis of the fatty acyl isethionate. The initial wormlike and spherical micellar composition may separate into an upper lamellar phase and a lower micellar phase. Lamellar phases are typically relatively high ordered, surfactant-rich phases with relatively less water and low density such that the lamellar phases float.

Hence, there is a need to provide a personal cleansing composition comprising a fatty acyl isethionate surfactant, being free of alkyl sulfate or alkyl ether sulfate type of surfactants and having an improved phase stability with a satisfactory consistent rheology profile.

There is a need to provide a personal cleansing composition comprising a fatty acyl isethionate surfactant, being free of alkyl sulfate or alkyl ether sulfate type of surfactants and having an acceptable or improved stability in terms of color and antimicrobial stabilities.

Also, there remains a need for a personal cleansing composition, which is effective at cleaning even while containing lower number of active surfactants than typical cleansing products, but also still possesses good esthetic properties such as good foam, and is thick and creamy in texture, is silky to the touch and affords conditioning.

### SUMMARY OF THE INVENTION

A personal cleansing composition is provided and comprises:
a) a surfactant system, wherein the surfactant system comprises:
   (i) from 0.1% to 5%, preferably from 0.5% to 4%, more preferably from 1% to 3%, most preferably from 1.5% to 2.5%, of a fatty acyl isethionate surfactant by weight of the composition;
   (ii) from 0.5% to 40%, preferably from 1% to 25%, more preferably from 5% to 15%, most preferably from 5.5% to 8.0% of a co-surfactant by weight of the composition;
      wherein the composition is free of alkyl sulfate or alkyl ether sulfate type of surfactants;
b) from 0.05% to 5%, preferably from 0.1% to 2%, more preferably from 0.5% to 1.5%, most preferably from 0.75% to 1.0% of a of a non-ionic surfactant by weight of the composition, wherein the non-ionic surfactant comprises a fatty alkanolamide;
c) from 0.01% to 2.0%, preferably from 0.05% to 1.5%, more preferably from 0.07% to 0.50%, most preferably from 0.1% to 0.15% of a hydrophobically modified ethoxylated methyl glucoside by weight of the composition;
d) from 0.01% to 1.0%, preferably from 0.02% to 0.4%, more preferably from 0.05% to 0.2%, most preferably from 0.05% to 0.1% of a preservative by weight of the composition; and
e) wherein the pH is from 5.5 to 7, preferably from 5.5 to 6.5, more preferably from 5.6 to 6.1.

A method for increasing the stability of a personal cleansing composition both in terms of phase, color and microbial stability is provided and comprises the step of forming a personal cleansing composition as described hereinafter.

### BRIEF DESCRIPTION OF THE DRAWINGS

While the specification concludes with claims particularly pointing out and distinctly claiming the present invention, it is believed that the same will be better understood from the following description read in conjunction with the accompanying drawings in which:

FIG. 1 is a graph representing the effect of a thickener and its relative concentration on enhancing the viscosity of a personal cleansing composition; and

FIG. 2 is a graph representing the effect of a thickening system at different levels of electrolyte being sodium chloride on enhancing the viscosity of a personal cleansing composition.

### DETAILED DESCRIPTION OF THE INVENTION

### Definitions of Terms

In this document, including in all embodiments of all aspects of the present invention, the following definitions apply unless specifically stated otherwise.

All percentages are by weight (w/w) of the composition, unless otherwise specified. “% wt.” means percentage by weight. References to ‘parts’ e.g. a mixture of 1 part X and 3 parts Y, is a ratio by weight. All ratios or percentages are weight ratios or weight percentages unless specifically stated otherwise.

An “active composition” is the composition absent water, and an “active ingredient” is the ingredient absent its water.

“QS” or “QSP” means sufficient quantity for 100% or for 100 g. +/− indicates the standard deviation. All ranges are inclusive and combinable. The number of significant digits conveys neither a limitation on the indicated amounts nor on the accuracy of the measurements. All numerical amounts are understood to be modified by the word “about”.

All measurements are understood to be made at 25° C. and at ambient conditions, where “ambient conditions” means at 1 atmosphere (atm) of pressure and at 65% relative humidity, unless otherwise stated. “Relative humidity” refers to the ratio (stated as a percent) of the moisture content of air compared to the saturated moisture level at the same temperature and pressure. Relative humidity can be measured with a hygrometer, in particular with a probe hygrometer from VWR® International.

Herein “min” means “minute” or “minutes”. Herein “mol” means mole. Herein “g” following a number means “gram” or “grams”. “Ex.” means “example”. All amounts as they pertain to listed ingredients are based on the active level and do not include carriers or by-products that may be included in commercially available materials.

Herein, “comprising” means that other steps and other ingredients can be in addition. “Comprising” encompasses the terms “consisting of” and “consisting essentially of”. The compositions, methods, uses, and processes of the present invention can comprise, consist of, and consist essentially of the elements and limitations of the invention described herein, as well as any of the additional or optional ingredients, components, steps, or limitations described herein. Embodiments and aspects described herein may comprise or be combinable with elements, features or components of other embodiments and/or aspects despite not being expressly exemplified in combination, unless an incompatibility is stated.

As used herein, the articles including “a” and “an” when used in a claim, are understood to mean “one or more” of what is claimed or described.

The terms “include,” “includes,” and “including,” as used herein are meant to be non-limiting.

Where amount ranges are given, these are to be understood as being the total amount of said ingredient in the composition, or where more than one species fall within the scope of the ingredient definition, the total amount of all ingredients fitting that definition, in the composition.

For example, if the composition comprises from 1% to 5% fatty alcohol, then a composition comprising 2% stearyl alcohol and 1% cetyl alcohol and no other fatty alcohol, would fall within this scope.

The amount of each particular ingredient or mixtures thereof described hereinafter can account for up to 100% (or 100%) of the total amount of the ingredient(s) in the composition.

The term “free of” as used herein means that the composition comprises 0% of an ingredient by weight of the composition, thus no detectable amount of the stated ingredient.

The term “substantially free of” as used herein means less than 1%, less than 0.8%, less than 0.5%, less than 0.3%, or less than an immaterial amount of by weight of the composition.

Herein “Comp. Ex.” or “C. Ex.” means comparative example; and “Ex.” means example.

The term “molecular weight” or “M.Wt.” as used herein refers to the weight average molecular weight unless otherwise stated. The weight average molecular weight can be measured by gel permeation chromatography (“GPC”).

The term “personal cleansing composition” as used herein refers to compositions intended for topical application to the hair and the skin, preferably to the skin, for cleansing.

The term “mixtures” as used herein is meant to include a simple combination of materials and any compounds that may result from their combination.

The term “room temperature” refers to a temperature of 25° C.

The term “rinse-off” as used herein means the intended product usage includes application to skin followed by rinsing and/or wiping the product from the skin within a few seconds to minutes of the application step. The product is generally applied and rinsed in the same usage event, for example, a shower or washing one's hands.

The term “derivative” as used herein refers to structures which are not shown but which one skilled in the art would understand are variations of the basic compound.

The methods as disclosed herein are cosmetic methods or non-therapeutic methods.

The objects of the present invention are to provide personal cleansing products, methods and uses of the products, the structures and the respective compositions as described in the Summary or as described hereinbelow for fulfilling the technical effects or goals as set out herein. These objects and other advantages as may be apparent to those skilled in the art can be achieved through the present invention, which is described in the above Summary of the Invention and Detailed Description of the invention and which is defined in the claims which follow.

### Benefits

Phase separation has been observed for personal cleansing compositions comprising a fatty acyl isethionate at 40° C. after 3 months (see C. Ex. 1 for an example). The mechanism of the phase separation might lie in the hydrolysis of the fatty acyl isethionate and other anionic surfactants such as a fatty acyl sarcosinate catalyzed in an acidic medium (e.g. pH at 5).

Increasing the pH would slow down the hydrolysis kinetics, however, at a neutral pH, the zero-shear viscosity of the personal cleansing composition may collapse. Only a small portion of a deprotonated form of anionic surfactant, e.g. fatty acyl sarcosinate, is needed to form a stable wormlike micellar phase without the addition of any electrolyte. The pH of the personal cleansing composition cannot be raised beyond the pKa of the anionic surfactant, otherwise, the viscosity peak is missed and the personal cleansing composition cannot meet the rheologic profile.

As there is a need to increase the pH to control the hydrolysis kinetics of the fatty acyl isethionate, a new combination of ingredients was needed to build the consistent viscosity of the personal cleansing composition.

It has been found that the personal cleansing composition needs to include from 0.05% to 5%, preferably from 0.1% to 2%, more preferably from 0.5% to 1.5%, most preferably from 0.75% to 1.0% of a of a non-ionic surfactant by weight of the composition, wherein the non-ionic surfactant comprises a fatty alkanolamide; and from 0.01% to 2.0%, preferably from 0.05% to 1.5%, more preferably from 0.07% to 0.50%, most preferably from 0.1% to 0.15% of a hydrophobically modified ethoxylated methyl gluco side by weight of the composition.

The desired viscosity for the personal cleansing composition can be achieved by elongating the micelles and this at a higher pH from 5.5 to 7, preferably from 5.5 to 6.5, more preferably from 5.6 to 6.1.

The choices of the non-ionic surfactant and the hydrophobically modified ethoxylated methyl glucoside, at specific levels and weight ratios, will be described in more details below.

Also, it has been observed that certain ingredients, for instance a fragrance component having hydrophobic materials might play a role in phase separation but also with discoloration. Without any addition of a fragrance component, already other ingredients such as the non-ionic surfactant and the hydrophobically modified ethoxylated methyl glucoside might also lead to discoloration of the personal cleansing composition. Thus, there was a need to investigate a suitable preservative system for providing an acceptable or improved color and antimicrobial stabilities.

The preservative may be selected from the group consisting of an alkali metal metabisulfite, an alkali metal sulfite, an alkali metal bisulfite, an alkali metal hydrosulfite, and mixtures thereof. The preservative can help for preventing any discoloration of the personal cleansing composition and for providing antimicrobial efficacy as explained more in details below.

### Surfactant System

A personal cleansing composition is provided and comprises a surfactant system, wherein the surfactant system comprises:
from 0.1% to 5%, preferably from 0.5% to 4%, more preferably from 1% to 3%, most preferably
from 1.5% to 2.5%, of a fatty acyl isethionate surfactant by weight of the composition;
from 0.5% to 40%, preferably from 1% to 25%, more preferably from 5% to 15%, most preferably from 5.5% to 8.0% of a co-surfactant by weight of the composition; and
wherein the composition is free of alkyl sulfate and/or alkyl ether sulfate type of surfactants.

The personal cleansing composition is free of alkyl sulfate and/or alkyl ether sulfate type of surfactant. Preferably, the personal cleansing composition does not comprise any alkyl sulfate which comprises C₁₂-C₁₈ alkyl sulfate and/or any alkyl ether sulfate including alkyl glyceryl ether sulfates.

The personal cleansing composition may not comprise any alkyl ether sulfates which are those having the formula:

RO(CH₂CH₂O)ₙSO₃M

wherein R is an alkyl or alkenyl having 8 to 18 carbons, preferably 12 to 18 carbons, n has an average value of greater than at least 0.5, preferably between 2 and 3; and M is a solubilizing cation such as sodium, potassium, ammonium or substituted ammonium.

The personal cleansing composition may not comprise any ammonium and sodium lauryl ether sulfates.

If the personal cleansing composition does contain alkyl sulfate and/or alkyl ether sulfate type of surfactant, its content of such a weight proportion of: alkyl sulfates or alkyl ether sulfate type surfactant is less than or equal to the sum of 0.6, more preferably less than or equal to the sum of 0.2, even more preferably equal to 0.

### Fatty Acyl Isethionate

The fatty acyl isethionate surfactant may be defined as an isethionate according to the general Formula (I):

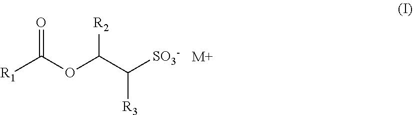

wherein R₁ is a saturated or unsaturated, straight or branched, alkyl or alkenyl chain with from 6 to 30 carbon atoms, preferably from 8 to 22 carbon atoms, more preferably from 9 to 18 carbon atoms, R₂ and R₃ are each independently H or (C₁-C₄) alkyl, and M⁺ is an alkali metal, preferably lithium, sodium, potassium; or M⁺ is an alkali-earth metal, preferably magnesium; or M⁺ is an ammonium or a substituted ammonium cation.

Preferably, R₁ may be a saturated or unsaturated, straight or branched alkyl or alkenyl, preferably an alkyl chain with from 6 to 30 carbon atoms, preferably from 8 to 22 carbon atoms, more preferably from 9 to 18 carbon atoms, R₂ and R₃ are H, and M⁺ is an alkali metal, preferably sodium, potassium; or M⁺ is an ammonium cation.

More preferably, R₁ may be a saturated or unsaturated, straight or branched alkyl chain with from 9 to 18 carbon atoms, R₂ and R₃ are H, and M⁺ is sodium or an ammonium cation.

Suitable fatty acyl isethionate surfactants may include the reaction product of fatty acids esterified with isethionic acid and neutralized with sodium hydroxide. Suitable fatty acids for isethionate surfactants can be derived from coconut oil or palm kernel oil, for instance. Additional examples of suitable isethionic anionic surfactants are described in U.S. Pat. Nos. 2,486,921; 2,486,922; and 2,396,278, each of which is incorporated herein by reference.

The fatty acyl isethionate surfactant may be selected from the group consisting of sodium lauroyl isethionate, sodium lauroyl methyl isethionate, sodium oleoyl isethionate, sodium oleoyl methyl isethionate, sodium stearoyl isethionate, sodium stearoyl methyl isethionate, sodium myristoyl isethionate, sodium myristoyl methyl isethionate, sodium palmitoyl isethionate, sodium palmitoyl methyl isethionate, sodium cocoyl isethionate, sodium cocoyl methyl isethionate, a blend of stearic acid and sodium cocoyl isethionate, ammonium cocoyl isethionate, ammonium cocoyl methyl isethionate, and mixtures thereof.

The fatty acyl isethionate surfactant may be preferably selected from the group consisting of sodium lauroyl isethionate, sodium myristoyl isethionate, sodium palmitoyl isethionate, sodium stearoyl isethionate, sodium oleoyl isethionate, sodium cocoyl isethionate, ammonium cocoyl isethionate, and mixtures thereof.

The fatty acyl isethionate surfactant may be more preferably selected from the group consisting of sodium lauroyl isethionate, sodium cocoyl isethionate, ammonium cocoyl isethionate, and mixtures thereof.

Corresponding commercial products are available, for example, from the company Innospec under the trade name “Iselux®” and from Clamant or Uniquema under the trade names “Hostapon®” or Arlatone®. Examples of other commercial fatty acyl isethionates that may be used can be Hostapon® surfactants from Clamant such as for sodium cocoyl isethionate: Hostapon® SCI-85C, Hostapon® SCI-78C, or a blend of stearic acid with sodium cocoyl isethionate: Hostapon® SCI-65C. Examples of other commercial fatty acyl isethionates that may be used can be “Jordapon®” surfactants from BASF such as Jordapon® CI prill or Jordapon® CI65; and sodium cocoyl isethionate from Yongan Daily Chemical Co. such as YA-SCI-85® or YA-SCI-65®.

Fatty acyl isethionates surfactants are typically prepared by the reaction of an isethionate salt such as metal or ammonium isethionate and an a saturated or unsaturated, straight or branched, alkyl or alkenyl chain fatty acid having from 6 to 30 carbon atoms, preferably from 8 to 22 carbon atoms, more preferably from 6 to 18 carbon atoms. Depending on the processing conditions used, the resulting fatty acyl isethionate surfactant can be a mixture of 45 to 95% by weight of fatty acyl isethionates and 0 to 40 wt. % of free fatty acids, in addition to isethionates salts, typically less than 5 wt. %, and trace (less than 2 wt. %) of other impurities, by total weight of the resulting fatty acyl isethionate surfactant. A mixture of aliphatic fatty acids may be used for the preparation of commercial fatty acyl isethionates surfactants.

The personal cleansing composition comprises a surfactant system. The surfactant system comprises from 0.1% to 5%, preferably from 0.5% to 4%, more preferably from 1% to 3%, most preferably from 1.5% to 2.5%, of a fatty acyl isethionate surfactant by weight of the composition. The concentrations mentioned here are total concentration ranges in case more than one fatty acyl isethionate surfactant is present. The specified ranges are provided by weight and relate to the total weight of the personal cleansing composition.

### Fatty Acyl Sarcosinate

The surfactant system may further comprise from 0.1% to 10%, preferably from 0.5% to 8%, more preferably from 1% to 5%, most preferably from 3.5% to 5%, of a fatty acyl sarcosinate surfactant by weight of the composition.

The fatty acyl sarcosinate surfactant may be a sarcosinate according to the general Formula (II):

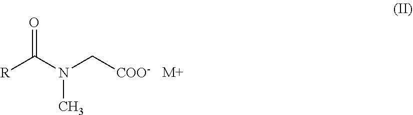

wherein R is a saturated or unsaturated, straight or branched or alkenyl, preferably alkyl chain with 7 to 17 carbon atoms, preferably with 9 to 13 carbon atoms and M⁺ is H, a sodium, potassium or ammonium cation.

The fatty acyl sarcosinate may be selected from the group consisting of sodium lauroyl sarcosinate, sodium cocoyl sarcosinate, sodium myristoyl sarcosinate, TEA-cocoyl sarcosinate, ammonium cocoyl sarcosinate, ammonium lauroyl sarcosinate, dimer dilinoleyl bis-lauroyl glutamate/lauroyl sarcosinate, lauroyl sarcosinate, isopropyl lauroyl sarcosinate, potassium cocoyl sarcosinate, potassium lauroyl sarcosinate, sodium oleoyl sarcosinate, sodium palmitoyl sarcosinate, TEA-lauroyl sarcosinate, TEA-oleoyl sarcosinate, TEA-palm kernel sarcosinate, and mixtures thereof. For instance, TEA-cocoyl sarcosinate is the triethanolamine salt of cocoyl sarcosine.

Preferably, the fatty acyl sarcosinate may be selected from the group consisting of sodium lauroyl sarcosinate, sodium myristoyl sarcosinate, sodium cocoyl sarcosinate, and mixtures thereof.

The weight ratio of the fatty acyl isethionate to the fatty acyl sarcosinate may be from 2:3 to 1:1, preferably 3:4 to 1:1. In that aspect, the phase stability of the personal cleansing composition can be enhanced by further elongating the micelles, thus building the viscosity of the personal cleansing composition.

### Co-Surfactant

The surfactant system comprises from 0.5% to 40%, preferably from 1% to 25%, more preferably from 5% to 15%, most preferably from 5.5% to 8.0% of a co-surfactant by weight of the composition.

The co-surfactant of the personal cleansing composition may include an amphoteric surfactant or a zwitterionic surfactant. Suitable amphoteric or zwitterionic surfactants can include those described in U.S. Pat. Nos. 5,104,646 and 5,106,609.

Amphoteric surfactants can include those that can be broadly described as derivatives of aliphatic secondary and tertiary amines in which an aliphatic radical can be straight or branched chain and wherein an aliphatic substituent can contain from 8 to 18 carbon atoms such that one carbon atom can contain an anionic water solubilizing group, e.g., carboxy, sulfonate, sulfate, phosphate, or phosphonate. Examples of compounds falling within this definition can be sodium 3-dodecyl-aminopropionate, sodium 3-dodecylaminopropane sulfonate, N-alkyltaurines such as the one prepared by reacting dodecylamine with sodium isethionate according to the teaching of U.S. Pat. No. 2,658,072, N-higher alkyl aspartic acids such as those produced according to the teaching of U.S. Pat. No. 2,438,091, and products described in U.S. Pat. No. 2,528,378.

The amphoteric surfactant included in the personal cleansing composition described herein may be preferably selected from the group consisting of sodium lauroamphoacetate, sodium cocoamphoacetate, disodium lauroamphoacetate, disodium cocodiamphoacetate, and mixtures thereof.

Zwitterionic surfactants suitable for use in the co-surfactants of the personal cleansing composition described herein may include those that are broadly described as derivatives of aliphatic quaternary ammonium, phosphonium, and sulfonium compounds, in which the aliphatic radicals can be straight or branched chains, and wherein one of the aliphatic substituents can contain from 8 to 18 carbon atoms and one can contain an anionic group, e.g., carboxy, sulfonate, sulfate, phosphate, or phosphonate.

Preferably, the co-surfactant may include a zwitterionic surfactant, wherein the zwitterionic surfactant comprises an alkyl betaine or an alkyl amidopropyl betaine.

Examples of betaine zwitterionic surfactants may include coco dimethyl carboxymethyl betaine, cocoamidopropyl betaine (CAPB), coco-betaine, lauryl amidopropyl betaine (LAPB), oleyl betaine, lauryl dimethyl carboxymethyl betaine, lauryl dimethyl alpha-carboxyethyl betaine, cetyl dimethyl carboxymethyl betaine, lauryl bis-(2-hydroxyethyl) carboxymethyl betaine, stearyl bis-(2-hydroxypropyl) carboxymethyl betaine, oleyl dimethyl gamma-carboxypropyl betaine, lauryl bis-(2-hydroxypropyl)alpha-carboxyethyl betaine, and mixtures thereof. Examples of sulfobetaines may include coco dimethyl sulfopropyl betaine, stearyl dimethyl sulfopropyl betaine, lauryl dimethyl sulfoethyl betaine, lauryl bis-(2-hydroxyethyl) sulfopropyl betaine and mixtures thereof.

More preferably, the zwitterionic surfactant may be selected from the group consisting of cocamidopropyl betaine, lauramidopropyl betaine, coco betaine, and mixtures thereof.

The surfactant system may most preferably comprise from 5.5% to 8.0% of a co-surfactant by weight of the composition, wherein the co-surfactant includes a zwitterionic surfactant, wherein the zwitterionic surfactant is selected from the group consisting of cocamidopropyl betaine, lauramidopropyl betaine, coco betaine, and mixtures thereof, preferably cocamidopropyl betaine. In that aspect, the phase stability of the personal cleansing composition can be further enhanced.

pH

The pH of the personal cleansing composition is from 5.5 to 7, preferably from 5.5 to 6.5, more preferably from 5.6 to 6.1. Increasing the pH can help for preventing hydrolysis of the fatty acyl isethionate and the fatty acyl sarcosinate. pH may be measured according to the Product pH Measurement Test Method, described hereafter.

A variety of compounds may be used to adjust the pH value of a composition. Such suitable compounds can include, but are not limited to, acetic acid, hydrochloric acid, sodium hydroxide, magnesium hydroxide, triethylamine, diethylamine, ethylamine, monoethanol amine, and any mixtures thereof. The personal cleansing composition may comprise greater than 0% to 2% of the pH adjusting agent by weight of the composition, preferably wherein the pH adjusting agent comprises citric acid.

Increasing the pH of the personal cleansing composition can help to prevent phase separation of the personal cleansing composition.

### Thickening System

The personal cleansing composition comprises from 0.05% to 5%, preferably from 0.1% to 2%, more preferably from 0.5% to 1.5%, most preferably from 0.75% to 1.0% of a of a non-ionic surfactant by weight of the composition. The non-ionic surfactant comprises a fatty alkanolamide.

The fatty alkanolamide may be selected from the group consisting of cocamide, cocamide methyl MEA, cocamide MEA, cocamide DEA, cocamide MIPA, lauramide MEA, lauramide DEA, lauramide MIPA, myristamide MEA, myristamide DEA, PEG-20 cocamide MEA, PEG-2 cocamide, PEG-3 cocamide, PEG-4 cocamide, PEG-5 cocamide, PEG-6 cocamide, PEG-7 cocamide, PEG-3 lauramide, PEG-5 lauramide, PEG-3 oleamide, PPG-2 cocamide, PPG-2 hydroxyethyl cocamide, PPG-2 hydroxyethyl isostearamide, soyamide DEA, oleamide MIPA, stearamide MEA, myristamide DEA, stearamide DEA, oleylamide DEA, tallowamide DEA, lauramide MIPA, tallowamide MEA, isostearamide DEA, isostearamide MEA, and mixtures thereof.

The fatty alkanolamide may be selected from the group consisting of cocamide, cocamide methyl MEA, cocamide MEA, cocamide DEA, cocamide MIPA, lauramide MEA, lauramide DEA, lauramide MIPA, myristamide MEA, myristamide DEA, soyamide DEA, oleamide MIPA, stearamide MEA, stearamide DEA, oleylamide DEA, tallowamide DEA, lauramide MIPA, tallowamide MEA, isostearamide DEA, isostearamide MEA, and mixtures thereof.

The fatty alkanolamide may be preferably selected from the group consisting of cocamide MEA, cocamide DEA, lauramide MEA, lauramide DEA, stearamide MEA, myristamide DEA, stearamide DEA, and mixtures thereof.

Most preferably, the fatty alkanolamide may be cocamide MEA.

The fatty alkanolamide can help to significantly boost the lather creaminess of the personal cleansing composition.

Also, the personal cleansing composition comprises from 0.01% to 2%, preferably from 0.05% to 1.5%, more preferably from 0.07% to 0.50%, most preferably from 0.1% to 0.15% of a hydrophobically modified ethoxylated methyl gluco side by weight of the composition.

The hydrophobically modified ethoxylated methyl glucoside may be selected from the group consisting of PEG-120 methyl glucose dioleate, PEG-120 methyl glucose trioleate, PEG-20 methyl glucose sesquistearate, PEG-20 methyl glucose distearate, PEG-80 methyl glucose laurate, PEG-20 methyl glucose sesquilaurate, PEG-120 methyl glucose triisostearate, and mixtures thereof.

Preferably, the hydrophobically modified ethoxylated methyl glucoside may be selected from the group consisting of PEG-120 methyl glucose dioleate, PEG-120 methyl glucose trioleate, PEG-20 methyl glucose sesquistearate, PEG-20 methyl glucose distearate, and mixtures thereof.

More preferably, the hydrophobically modified ethoxylated methyl glucoside may be selected from the group consisting of PEG-120 methyl glucose dioleate, PEG-120 methyl glucose trioleate, and mixtures thereof. Most preferably, the hydrophobically modified ethoxylated methyl glucoside may comprise PEG-120 methyl glucose trioleate.

The hydrophobically modified ethoxylated methyl glucoside is a naturally-derived thickener for providing mildness in the personal cleansing composition.

The hydrophobically modified ethoxylated methyl glucoside is a methyl glucose ether which has been esterified with a fatty acid, such as oleic acid or stearic acid for instance.

The hydrophobically modified ethoxylated methyl glucoside can also help to adjust, typically by reducing the lather amount of the personal cleansing composition.

When the personal cleansing composition comprises the fatty alkanolamide and the hydrophobically modified ethoxylated methyl glucoside as recited just above, the phase stability of the personal cleansing composition is improved. The fatty alkanolamide and the hydrophobically modified ethoxylated methyl glucoside could efficiently build the personal cleansing composition at the desired and consistent viscosity. The fatty alkanolamide and the hydrophobically modified ethoxylated methyl glucoside can interact by elongating from spherical micelle to wormlike micelle made of the fatty acyl isethionate surfactant, or the fatty acyl isethionate surfactant and fatty acyl sarcosinate.

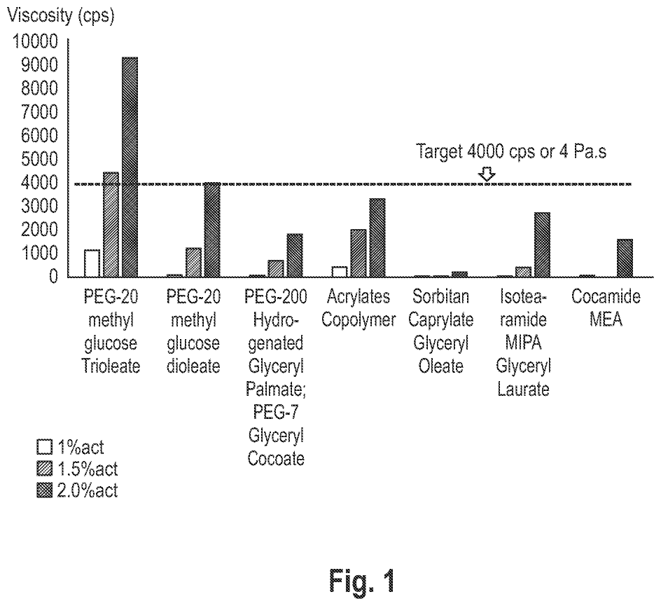

First, different thickeners were preliminary assessed. FIG. 1 is a graph representing the effect of a thickener with its relative concentration on enhancing the viscosity of a personal cleansing composition such as C. Ex. 1 as an example as detailed in the Example section. The personal cleansing composition shall have a viscosity target around 4 Pa·s (4000 cps). At 2 wt. % level, hydrophobically modified ethoxylated methyl glucosides such as of PEG-120 methyl glucose dioleate or PEG-120 methyl glucose trioleate were found to significantly increase the viscosity of the personal cleansing composition compared to other types of thickeners at the same levels such as acrylates copolymer, PEG-200 hydrogenated glyceryl palmate with PEG-7 glyceryl cocoate, sorbitan caprylate with glyceryl oleate, or isostearamide MIPA with glyceryl laurate.

Also, a fatty alkanolamide like cocamide MEA showed a good response.

Other thickeners not shown in FIG. 1 led to an unacceptable precipitated gel. Unsuitable thickeners were PEG-14M, PEG-2M, PQ-10, hydroxypropyl methylcellulose, or sodium carboxymethyl cellulose.

Cationic polymers such as Polyquaternium-10 were too hydrophilic and provides a relatively weak interaction with the surfactant system.

Anionic polymers namely acrylates copolymer also provided a too weak interaction with the surfactants of the composition due to a too high ionic strength.

Hydrophobically modified ethoxylated methyl glucosides are assumed to interact efficiently with the surfactant micelles due to the relatively high number of hydrophobic blocks and the compatibility with the surfactant system.

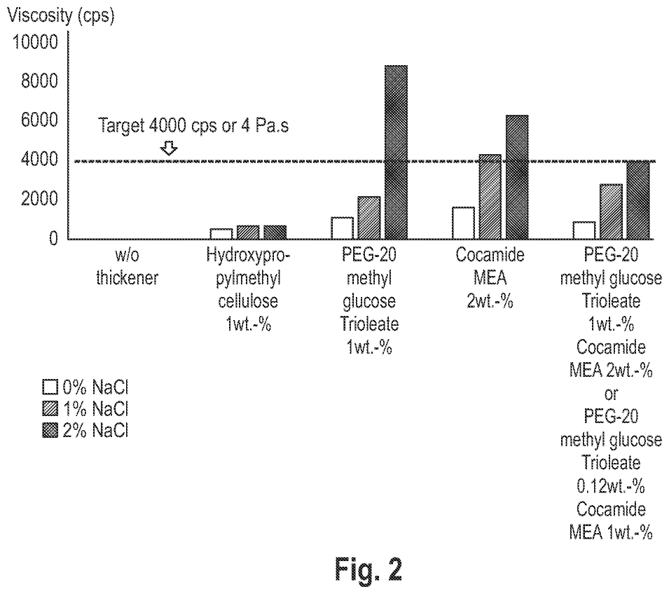

FIG. 2 is a graph representing the effect of a thickening system at different levels of electrolyte being sodium chloride on enhancing the viscosity of a personal cleansing composition such as C. Ex. 1 as an example as detailed in the Example section.

For different levels of sodium chloride at 0%, 1% and 2% by weight of the composition, it has been found that the combination of 2 wt. % cocamide MEA and 1 wt. % PEG-20 methyl glucose trioleate was found to provide enough viscosity.

In particular, the same result was obtained for a combination of 1 wt. % cocamide MEA and 0.12 wt. % PEG-20 methyl glucose trioleate. The weight ratio of the fatty alkanolamide to the hydrophobically modified ethoxylated methyl glucoside was then 8.3.

The viscosity target was reached with the addition of 2 wt. % of sodium chloride. With 1 wt. % of sodium chloride, the viscosity was between 2000 and 4000 cps which was acceptable.

Interestingly, the results were also observed in the Example section below without the addition of any electrolyte such as sodium chloride. Hence, any addition of electrolyte is merely an option to adjust further the viscosity of the personal cleansing composition if necessary.

Also, it was possible to decrease the levels of both fatty alkanolamide and the hydrophobically modified ethoxylated methyl glucoside with a certain weight ratio to provide the desired viscosity of the composition.

Thus, the weight ratio of the fatty alkanolamide to the hydrophobically modified ethoxylated methyl glucoside may range from 2 to 12, preferably from 5 to 10, more preferably from 8 to 9.

Alternatively, the fatty alkanolamide may comprise cocamide MEA and the hydrophobically modified ethoxylated methyl glucoside may be selected from the group consisting of PEG-120 methyl glucose dioleate, PEG-120 methyl glucose trioleate, and mixtures thereof, wherein the weight ratio of the fatty alkanolamide to the hydrophobic ally modified ethoxylated methyl glucoside ranges from 5 to 12, preferably from 7 to 10, more preferably from 8 to 9.

Preferably, the fatty alkanolamide may comprise cocamide MEA and the hydrophobically modified ethoxylated methyl glucoside may comprise PEG-120 methyl glucose trioleate, wherein the weight ratio of the fatty alkanolamide to the hydrophobically modified ethoxylated methyl glucoside ranges from 5 to 12, preferably from 7 to 10, more preferably from 8 to 9.

### Preservative

Increasing the pH can have a detrimental impact on the preservative system. Salicylic acid at a pH between 5.5 to 7 may be deprotonated and might not act as a robust preservative.

Also, the addition of a fatty alkanolamide, e.g. cocamide MEA and a hydrophobically modified ethoxylated methyl glucoside as set out above might lead to discoloration of the personal cleansing composition.

Hence, the personal cleansing composition includes from 0.01% to 1.0%, preferably from 0.02% to 0.4%, more preferably from 0.05% to 0.2%, most preferably from 0.05% to 0.1% of a preservative by weight of the composition.

The preservative may be selected from the group consisting of an alkali metal metabisulfite, an alkali metal sulfite, an alkali metal bisulfite, an alkali metal hydrosulfite, and mixtures thereof. An alkali metal may be lithium, sodium or potassium.

The preservative may be selected from the group consisting of sodium metabisulfite, sodium sulfite, sodium hydrosulfite, potassium sulfite, sodium bisulfite, potassium bisulfite and mixtures thereof. Preferably, the preservative may comprise sodium bisulfite.

The personal cleansing composition may include from 0.05% to 0.2%, preferably from 0.05% to 0.1% of a preservative by weight of the composition, wherein the preservative is selected from the group consisting of sodium metabisulfite, sodium sulfite, sodium hydrosulfite, potassium sulfite, sodium bisulfite, potassium bisulfite and mixtures thereof, more preferably wherein the preservative comprises sodium bisulfite.

The preservative may further include a salicylate salt and a benzoate salt, wherein a total amount of the salicylate salt and the benzoate salt is from 0.2% to 0.9%, preferably from 0.5% to 0.85%, more preferably from 0.75% to 0.85%, by weight of the composition.

The weight ratio of the salicylate salt to the benzoate salt may be from 1:1.20 to 1:1.10, preferably from 1:1.175 to 1:1.125.

The personal cleansing composition may include from 0.05% to 0.2%, preferably from 0.05% to 0.1% of a preservative by weight of the composition, wherein the preservative is selected from the group consisting of sodium metabisulfite, sodium sulfite, sodium hydrosulfite, potassium sulfite, sodium bisulfite, potassium bisulfite and mixtures thereof, more preferably wherein the preservative comprises sodium bisulfite; and further a salicylate salt and a benzoate salt, wherein a total amount of the salicylate salt and the benzoate salt is from 0.2% to 0.9%, preferably from 0.5% to 0.85%, more preferably from 0.75% to 0.85%, by weight of the composition.

The salicylate salt may be sodium salicylate. The benzoate salt may be sodium benzoate.

The preservative as described hereinbefore can also prevent any discoloration of the personal cleansing composition when comprising a betaine or a fragrance component. Indeed, some fragrance component may contain aldehyde and amines that can react together and might provide discoloration over time.

Also, the preservative can show relatively strong antimicrobial efficacy at the recited pH from 5.5 to 7, especially at a pH of 6.4.

The preservative can also contribute in some extents to increase the antimicrobial preservation of the personal cleansing composition.

### Electrolyte

The personal cleansing composition may further comprise from 0.05 to 5%, preferably from 0.5% to 4%, more preferably from 1.0% to 2% of an electrolyte by weight of the composition. The addition of an electrolyte can help to elongate the micelles of the surfactant system and to improve further the viscosity of the composition if needed.

The electrolyte may be selected from the group of sodium or potassium citrate, calcium chloride, calcium bromide, zinc chloride, barium chloride, calcium nitrate, potassium chloride, sodium chloride, potassium iodide, sodium bromide, ammonium bromide, sodium sulfate, and mixtures thereof.

The electrolyte may be preferably selected from the group of sodium or potassium citrate, calcium chloride, potassium chloride, sodium chloride, and mixtures thereof.

Most preferably, the personal cleansing composition may further comprise from 1.0% to 2% of an electrolyte by weight of the composition, wherein the electrolyte is sodium chloride.

### Optional Ingredients

As can be appreciated, the compositions described herein may include a variety of optional components to tailor the properties and characteristics of the composition. As can be appreciated, suitable optional components are well known and can generally include any components which are physically and chemically compatible with the essential components of the compositions described herein. Optional components should not otherwise unduly impair product stability, aesthetics, or performance. Individual concentrations of optional components can generally range from 0.001% to 10%, by weight of the composition. Optional components can be further limited to components which will not impair the clarity of a translucent composition.

Still, the personal cleansing composition may not include or may be free of direct dyes, oxidative dyes, parabens, or mixtures thereof.

Optional components may include, but are not limited to, conditioning agents (including hydrocarbon oils, fatty esters, silicones), cationic polymers, anti-dandruff actives, and chelating agents. Additional suitable optional ingredients include but are not limited to particles, anti-microbials, foam boosters, anti-static agents, moisturizing agents, propellants, self-foaming agents, pearlescent agents, opacifiers, sensates, suspending agents, solvents, diluents, anti-oxidants, vitamins, and mixtures thereof.

### Fragrance Component

The personal cleansing composition may comprise from 0.01 wt. % to 2 wt. % of a fragrance component by weight of the composition, preferably from 0.1 wt. % to 1.75 wt. % of a fragrance component by weight of the composition, more preferably from 0.5 wt. % to 1.6 wt. % of a fragrance component by weight of the composition, even more preferably from 0.8 wt. % to 1.0 wt. % of a fragrance component by weight of the composition.

Typically the fragrance component may be a blend of perfumes and aroma chemicals. As used herein, “fragrance” is used to indicate any odoriferous material.

A wide variety of chemicals are known as fragrances, including alcohols, aldehydes, ketones, and esters. Non-limiting examples of the fragrances useful herein include pro-fragrances such as acetal pro-fragrances, ketal pro-fragrances, ester pro-fragrances, hydrolyzable inorganic-organic pro-fragrances, and mixtures thereof. The fragrances may be released from the pro-fragrances in a number of ways. For example, the fragrance may be released as a result of simple hydrolysis, or by a shift in an equilibrium reaction, or by a pH-change, or by enzymatic release.

The fragrances herein may be relatively simple in their chemical make-up, comprising a single chemical, or may comprise highly sophisticated complex mixtures of natural and synthetic chemical components, all chosen to provide any desired odor.

The fragrances may have a boiling point (BP) of 500° C. or lower, 400° C. or lower, or 350° C. or lower. The BP of many fragrances are disclosed in Perfume and Flavor Chemicals (Aroma Chemicals), Steffen Arctander (1969). The C log P value of the fragrances may be 0.1 or greater, 0.5 or greater, 1.0 or greater, and 1.2 or greater. As used herein, “C log P” means the logarithm to the base 10 of the octanol/water partition coefficient. The C log P may be readily calculated from a program called “C LOG P” which is available from Daylight Chemical Information Systems Inc., Irvine Calif., USA. Octanol/water partition coefficients are described in more detail in U.S. Pat. No. 5,578,563.

Suitable fragrances are also disclosed in U.S. Pat. Nos. 4,145,184, 4,209,417, 4,515,705, and 4,152,272. Non-limiting examples of fragrances include animal fragrances such as musk oil, civet, castoreurn, ambergris, plant fragrances such as nutmeg extract, cardomon extract, ginger extract, cinnamon extract, patchouli oil, geranium oil, orange oil, mandarin oil, orange Hower extract, cedarwood, vetyver, lavandin, ylang extract, tuberose extract, sandalwood oil, bergamot oil, rosemary oil, spearmint oil, peppermint oil, lemon oil, lavender oil, citronella oil, chamomille oil, clove oil, sage oil, neroli oil, labdanum oil, eucalyptus oil, verbena oil, mimosa extract, narcissus extract, carrot seed extract, jasmine extract, olibanum extract, rose extract, and mixtures thereof.

Other examples of suitable fragrances include, but are not limited to, chemical substances such as acetophenone, adoxal, aldehyde C-12, aldehyde C-14, aldehyde C-18, allyl caprylate, ambroxan, amyl acetate, dimethylindane derivatives, α-amylcinnamic aldehyde, anethole, anisaldehyde, benzaldehyde, borneol, butyl acetate, camphor, carbitol, cinnamaldehyde, cinnamyl acetate, cinnamyl alcohol, cis-3-hexanol and ester derivatives, cis-3-bexenyl methyl carbonate, citral, citronnellol and ester derivatives, cumin aldehyde, cyclamen aldehyde, cyclogalbanate, damascones, decalactone, decanol, estragole, dihydromyrcenol, dimethyl benzyl carbinol, 6,8-dimethyl-2-nonanol, dimethyl benzyl carbinyl butyrate, ethyl acetate, ethyl isobutyrate, ethyl butyrate, ethyl propionate, ethyl caprylate, ethyl cinnamate, ethyl hexanoate, ethyl valerate, ethyl vanillin, eugenol, exaltoiide, fenchone, fruity esters such as ethyl 2-methyl butyrate, galaxolide, geraniol and ester derivatives, helional, 2-heptonone, hexenol, α-hexylcinnamic aldehyde, hydroxycitronellal, indole, isoamyl acetate, isoeugenol acetate, ionones, isoeugenol, isoamyl iso-valerate, iso E super, limonene, linalool, lilial, linalyl acetate, lyral, majantol, mayol, melonal, menthol, p-methylacetophenone, methyl anthranilate, methyl cedrylone, methyl dibydrojasmonate, methyl eugenol, methyl ionone, methyl-α-naphthyl ketone, methylphenylcarbinyl acetate, mugetanol, γ-nonalactone, octanal, phenyl ethyl acetate, phenylacetaldehyde dimethyl acetate, phenoxyethyl isobutyrate, phenyl ethyl alcohol, pinenes, sandalore, santaiol, stemone, thymol, terpenes, triplal, triethyl citrate, 3,3,5-trimethylcyclohexanol, γ-undecalactone, undecenal, vanillin, veloutone, verdox, and mixtures thereof.

In the personal cleansing composition, the fragrance component may comprise ketone and/or aldehyde fragrance components. In addition, the fragrance component may further comprise any fragrances as set out just above.

Ketone fragrance components may be selected from alicyclic ketones such a β-ionone, terpene ketones such as 1-carvone, and macrocyclic ketones such as cyclopentadecanone.

Aldehyde fragrance components may be selected from fatty aldehydes such as 2,6-nonadienal, terpene aldehydes such as citral, and aromatic aldehydes such as α-hexylcinnamic aldehyde, cinnamaldehyde.

Thus, preferably the fragrance component may comprise ketone and/or aldehyde fragrance components, wherein the ketone and/or aldehyde fragrance components may be selected from the group consisting of acetophenone, adoxal, aldehyde C-12, aldehyde C-14, aldehyde C-18, α-amylcinnamic aldehyde, anisaldehyde, benzaldehyde, camphor, cinnamaldehyde, citral, cumin aldehyde, cyclamen aldehyde, damascones, fenchone, helional, 2-heptonone, α-hexylcinnamic aldehyde, hydroxycitronellal, ionones, lilial, lyral, melonal, p-methylacetophenone, methyl cedrylone, methyl ionone, methyl-α-naphthyl ketone, γ-nonalactone, octanal, phenylacetaldehyde dimethyl acetate, triplal, γ-undecalactone, undecenal, vanillin, veloutone, and mixtures thereof.

### Method

A method of increasing the stability of a personal cleansing composition both in terms of phase, color and microbial stability is provided and comprises the step of forming a personal cleansing composition as set out hereinbefore.

### Forms and Uses

### Product Form

The personal cleansing composition may be presented in typical personal cleansing formulations. They may be in the form of solutions, dispersion, emulsions, foams, and other delivery mechanisms. The personal cleansing composition may be a rinse-off composition.

The personal cleansing composition may be extrudable or dispensable from a single chamber package. The personal cleansing compositions can be in the form of liquid, semi-liquid, cream, lotion or gel, or solid compositions intended for topical application to skin.

Examples of personal cleansing compositions can include but are not limited to body wash, moisturizing body wash, foaming body wash, shower gels, a shower or bath cream, skin cleansers, cleansing milks, body wash, in shower body moisturizer, gel, emulsion, oil, mousse or spray.

The personal cleansing composition may be not in the form of a liquid hand wash or a liquid hand sanitizer.

The product forms contemplated for purposes of defining the personal cleansing compositions and methods are rinse-off formulations by which it is meant that the product is applied topically to the skin and then subsequently (i.e., within minutes) rinsed away with water, or otherwise wiped off using a substrate or other suitable removal means.

### Uses

The personal cleansing composition as set out hereinabove may be used for improving the lather of the composition.

The personal cleansing composition as set out hereinabove may be used for suspending benefits agents selected from the group consisting of hair care and skin care benefit agents, particulates, particles, preferably silica and titanium oxide, microcapsules, oils, droplets, pigments or coloring agents, opacifiers, pearlescent agents, feel modifiers, oil absorbers, skin protectants, matting agents, friction enhancers, slip agents, conditioning agents, exfoliants, odor absorbers, or cleaning enhancers, and mixtures thereof.

The personal cleansing composition can advantageously provide relatively improved ecotoxic or ecologically friendly environmental profile.

The personal cleansing composition can help to provide good esthetic properties such as good foam, and is thick and creamy in texture, is silky to the touch and affords conditioning.

### TEST METHODS

It is understood that the Test Methods that are disclosed in the Test Methods Section of the present application should be used to determine the respective values of the parameters of Applicants' invention as such invention is described and claimed herein.

### Cone/Plate Viscosity Measurement

The viscosity of the personal cleansing composition is measured by a Cone/Plate Brookfield DV12T, by Brookfield Engineering Laboratories, Stoughton, MA. The cone used (Spindle CPA-41z) has a diameter of 24 mm and 3° angle. The viscosity is determined using a steady state flow experiment at constant shear rate of 2 s⁻¹ and at temperature of 26.5° C. The sample size is 2.5 mL.

### Micro Hostility Measurement

### Test Microorganisms

Use cultures of the following microorganisms: Candida albicans, Aspergillus niger, Escherichia coli, Pseudomonas aeruginosa, Burkholderia cepacian, Klebsiella pneumoniae, Enterobacter gergoviae, Serratia marcescens and Staphylococcus aureus. The viable microorganisms used in the test must not be more than five passages removed from the original culture. For purposes of the test, one passage is defined as the transfer of microorganisms from an established culture to a fresh medium. All transfers are counted. In the case of microorganisms maintained by seed-lot techniques, each cycle of freezing, thawing, and revival in the fresh medium is taken as one transfer. A seed-stock technique should be used for long-term storage of cultures.

TABLE 1
Test Microorganisms

| Product category | Inoculum Type | Microorganism |
|---|---|---|
| Non-regulated | Yeast/Mold | Escherichia coli |
| product | pool | Staphylococcus aureus |
| | | Pseudomonas aeruginosa |
| | | Burkholderia cepacia |
| | | Klebsiella pneumoniae |
| | | Enterobacter gergoviae |
| | | Serratia marcescens |
| | Bacterial Pool | Candida albicans |
| | | Aspergillus brasiliensis |

### Preparation of Inoculum

Preparatory to the test, inoculate the surface of a suitable volume of solid agar medium from a recently revived stock culture of each of the specified microorganisms.

To harvest the bacterial and C. albicans cultures, use sterile saline solution to obtain a microbial count between 1×10⁵ and 1×10⁷ colony-forming units (cfu) per mL. To harvest the cells of A. brasiliensis, use sterile saline solution containing 0.05% of polysorbate 80, and add sufficient sterile saline solution to obtain a count in the range of 1×10⁷ to 1×10⁸ cfu/mL.

To create pooled inoculum, mix equal parts of each bacterium or Yeast and mold required for the pool described in Table 1.

### Procedure

The test can be conducted in two sterile, capped bacteriological containers of suitable size into which a sufficient volume of product has been transferred. Inoculate each container with one of the prepared and standardized inoculums, and mix. The volume of the suspension inoculum used is 1.0% of the volume of the product. The concentration of test microorganisms that is added to the product are such that the final concentration of the test preparation after inoculation is between 1×10⁵ and 1×10⁷ cfu per mL of the product.

The initial concentration of viable microorganisms in each test preparation is estimated based on the concentration of microorganisms in each of the standardized inoculum as determined by the plate-count method.

Incubate the inoculated containers at 20-25° C. Record any changes observed in appearance at these intervals. Determine by the plate-count procedure the number of cfu present in each test preparation for the applicable intervals. Incorporate an inactivator (neutralizer) of the specific antimicrobial in the plate count or in the appropriate dilution prepared for plating. Using the calculated concentrations of cfu per mL present at the start of the test, calculate the change in log 10 values of the concentration of cfu per mL for each microorganism at the applicable test intervals, and express the changes in terms of log reductions.

### Product pH Measurement

First, calibrate the Metler TOLEDO pH meter. Do this by turning on the pH meter and waiting for 30 seconds. Then, take the electrode out of the storage solution, rinse the electrode with reversed osmosis (RO) water, and carefully wipe the electrode with a scientific cleaning wipe, such as a Kimwipe®. Submerse the electrode in the pH 4 buffer and press the calibrate button. Wait until the pH icon stops flashing. Rinse the electrode with RO water and carefully wipe the electrode with a scientific cleaning wipe. Then submerse the electrode into the pH 7 buffer and wait until the pH icon stops flashing. Rinse the electrode with RO water and carefully wipe with a scientific cleaning wipe. Then, submerse the electrode into the pH 9 buffer and wait until the pH icon stops flashing. Rinse the electrode with distilled RO water and carefully wipe with a scientific cleaning wipe. Now the pH meter is calibrated and can be used to test the pH of a solution.

### Color Variation Test Method

The color variation test method was used to monitor color stability and to assess whether discoloration has been prevented or reduced. In the color variation test method, the compositions to be assessed were stamped into dimension of (62 mm*37 mm*16.5 mm). The color of the composition to be tested was evaluated using a benchtop spectrophotometer (a X-rite Ci 7800 instrument) via transmission mode. The composition to be tested was placed between two aperture plates within the measurement chamber. The incident light transmits through the composition to be tested and the absorbance of the lights was recorded over the wavelength range of 360-780 nm. The resulting curve was converted to the various color scale, L*, a* and b* values.

L* represents the lightness of the color, a* indicates the green/red color axis and b* the blue/yellow color axis.

Analysis of color was based on individual color components in the Hunter L* a* b* color space, wherein:

L*=lightness (L*=0 yields black and L*=100 indicates diffuse white)

a*=red/green (a*, negative values indicate green while positive values indicate red)

b*=yellow to blue (b*, negative values indicate blue and positive values indicate yellow)

To evaluate the color stability of the composition, color of the composition was measured when it is freshly made and after being aged at 75° C. for 4 hours.

Given ΔL* Δa* Δb*, the total difference or distance with Hunter L* a scale can be stated as a single value, known as ΔE*.

ΔE*=((L*₁−L*₂)²+(a*₁−a*₂)²+(b*₁−b*₂)²)^{1/2}.

wherein

L*₁, a*₁, b*₁ are the values for the fresh made composition (“initial”); L*₂, a*₂, b*₂ are the values for the aged composition (“final”).

A higher ΔE* value indicates larger color shift from initial value, which is unfavorable meaning that more color has been formed within the personal cleansing composition.

### EXAMPLES

The following examples further describe and demonstrate embodiments within the scope of the present invention. The examples are given solely for the purpose of illustration and are not to be construed as limitations of the present invention, as many variations thereof are possible without departing from the spirit and scope of the invention. Where applicable, ingredients are identified by chemical or CTFA name, or otherwise defined below.

The following examples were prepared:

### Compositions (wt. %)

| Ingredients (wt. %) | C. Ex. 1 | C. Ex. 2 | C. Ex. 3 | C. Ex. 4 | Ex. 1 | Ex. 2 |
|---|---|---|---|---|---|---|
| Sodium cocoyl isethionate¹ | 2.00 | 2.00 | 2.00 | 2.00 | 2.00 | 2.00 |
| Sodium lauroyl sarcosinate² | 4.30 | 4.30 | 4.30 | 4.30 | 4.30 | 4.30 |
| Cocamidopropyl betaine³ | 7.75 | 7.75 | 7.75 | 7.75 | 7.75 | 7.75 |
| Cocamide MEA⁴ | — | — | 1.00 | 1.00 | 1.00 | 1.00 |
| PEG-120 methyl glucose | — | — | — | — | 0.12 | 0.12 |
| trioleate⁵ | | | | | | |
| Ratio CMEA/PEG-120 | — | — | — | — | 8.3 | 8.3 |
| methyl glucose trioleate | | | | | | |
| Sodium salicylate⁶ | 0.40 | 0.40 | 0.40 | 0.40 | 0.40 | 0.40 |
| Sodium benzoate⁷ | 0.45 | 0.45 | 0.45 | 0.45 | 0.45 | 0.45 |
| Citric acid powder⁸ | q.s. | q.s. | q.s. | q.s. | q.s. | q.s. |
| | pH 5.0 | pH 6.1 | pH 6.1 | pH 6.1 | pH 6.1 | pH 6.1 |
| Sodium bisulfite⁹ | — | — | — | 0.10 | — | 0.10 |
| Fragrance | 1.0 | 1.0 | 1.0 | 1.0 | 1.0 | 1.0 |
| water | Balance | Balance | Balance | Balance | Balance | Balance |
| | to 100 | to 100 | to 100 | to 100 | to 100 | to 100 |
| Initial viscosity (Pa · s) | 11.31 | 0.049 | 3.09 | 0.68 | 4.44 | 3.95 |
| Viscosity after 3 weeks | 0.61 | 0.122 | 1.96 | 1.19 | 9.06 | 8.11 |
| at 60° C. (Pa · s) | | | | | | |
| Stability results | Phase | No | No | No | No | No |
| | separation | separation | separation | separation | separation | separation |
| Color stability ΔE* | 2.0 | 3.2 | 3.7 | 0.18 | 3.5 | 0.49 |
| Microhostility | 4.7 | 3.0 | 2.8 | 4.7 | 2.1 | 3.0 |
| (log reduction CFU) | | | | | | |

Definitions of Components
¹Sodium cocoyl isethionate sulfate; Supplier Clariant
²Sodium lauroyl sarcosinate; Supplier Tinci
⁴Cocamidopropyl Betaine; Supplier Tinci
⁵Glucamate ™ LT; Supplier Lubrizol Advanced Materials
⁶Sodium salicylate; Supplier JQC Huayn Pharmaeutical Co Ltd.
⁷Sodium benzoate; Supplier Wuhan Youji Industries
⁸Citric acid powder; Supplier Yixing Union Biochemical
⁹Sodium bisulfite: Supplier Suzhou Boyang Chemical
q.s.: sufficient quantity

### Results:

### Phase Stability and Enhanced Viscosity

The personal cleansing composition shall have an initial viscosity around 4 Pa·s (4000 cps) and below 15 Pa·s (15000 cps) but not below 3 Pa·s (3000 cps) after 3 weeks at 60° C.

C. Ex. 1 is a personal cleansing composition comprising sodium cocoyl isethionate, sodium lauroyl sarcosinate and cocamidopropyl betaine at a pH 5.0. After 3 weeks at 60° C., phase separation has been observed with a drop of the initial viscosity.

The top floating layer is a lamellar phase layer which is relatively highly ordered and comprises the above surfactants with relatively less water. As their density is lower, the lamellar phase floats and is the upper layer. The micellar phase is the lower layer.

The free fatty acids originating from sodium cocoyl isethionate or sodium lauroyl sarcosinate due to the respective hydrolysis of the anionic surfactants may contribute for the phase transition and phase separation from a micellar phase to a lamellar phase.

Initially, the hydrolysis of sodium cocoyl isethionate or sodium lauroyl sarcosinate causes the viscosity to increase due to the elongation of the wormlike micelles. After the generation of a certain amount of free fatty acid, however, the wormlike and spherical micelles transition from being wormlike micelles to bigger, more complex aggregates, such as disc-like micelles or gel networks. At that moment, these structures decrease the number of entanglements, reduce the resistance to flow, and increase fluidity of the system, resulting in a viscosity drop. A lamellar phase separated from a micellar phase.

When pH is increased from 5.0 to 6.1 in C. Ex. 2, the kinetics of the hydrolysis of sodium cocoyl isethionate and sodium lauroyl sarcosinate have been slowed down. No phase separation was observed. However, still the rheological properties of the personal cleansing composition was not satisfactory to be dispensed to the consumer.

In C. Ex. 3, 1 wt. % of cocamide MEA has been added which improved the viscosity properties of the personal cleansing composition.

Ex. 1 and Ex. 2 showed that the combination of cocamide MEA as a fatty alkanolamide and PEG-120 methyl glucose trioleate as a hydrophobically modified ethoxylated methyl glucoside could build the texture of the composition at desired enhanced viscosity. It has been found that the elongation from spherical micelles to wormlike micelle could be demonstrated by Cryo-TEM and NMR.

Similar results were obtained when replacing in Ex. 1 or Ex. 2 PEG-120 methyl glucose trioleate with PEG-120 methyl glucose dioleate supplied by Lubrizol as Glucamate™ DOE-120. The objective was to reach an initial viscosity target around 4 Pa·s.

As shown preliminary in FIG. 1, a hydrophobically modified ethoxylated methyl glucoside such as PEG-120 methyl glucose trioleate supplied by Lubrizol as Glucamate™ LT when used alone without any fatty alkanolamide was also found better in enhancing the composition texture at desired enhanced viscosity versus:
PEG-200 hydrogenated glyceryl palmate/PEG-7 glyceryl cocoate supplied by Evonik as ANTIL® 200 MB;
Acrylates Copolymer supplied by Lubrizol as Carbopol® Aqua SF-2;
Sorbitan caprylate/glyceryl oleate supplied by Evonik as TEGO® REMO 95 MB; or
isostearamide MIPA/glyceryl laurate supplied by Evonik as SPA 80.

Especially, cellulose type polymer such as hydroxypropyl methylcellulose, or sodium carboxymethylcellulose failed to build the desired viscosity and precipitation could be observed.

Even at different electrolyte levels, hydroxypropyl methylcellulose was not a thickener of interest, as shown in FIG. 2.

When a mixture of a fatty alkanolamide such as cocamide MEA and a hydrophobically modified ethoxylated methyl glucoside such as PEG-120 methyl glucose trioleate was used, the desired viscosity of the personal cleansing composition could be achieved with a weight ratio of the fatty alkanolamide to the hydrophobically modified ethoxylated methyl glucoside of 2 or 8.3 (See FIG. 2).

Hence, it has been found that the weight ratio of the fatty alkanolamide to the hydrophobically modified ethoxylated methyl glucoside ranges from 2 to 12, preferably from 5 to 10, more preferably from 8 to 9 especially in Example 1 with a weight ratio of 8.3.

### Antimicrobial and Discoloration Stabilities

In terms of color stability, after 3 weeks at 60° C., the personal cleansing composition shall have a ΔE* the smallest as possible.

Ex. 1 had a similar ΔE* than C. Ex. 2. Ex. 1 was an acceptable personal cleansing composition.

There was an additional need to further prevent discoloration after 3 weeks at 60° C.

When sodium bisulfite is added in Ex. 2 versus Ex. 1, a decrease of ΔE* is obtained showing that discoloration has been prevented.

As a dual function, sodium bisulfite as a preservative could not only effectively prevent discoloration of the personal cleansing composition but also showed strong antimicrobial efficacy at the claimed pH.

C. Ex. 2 has an increased pH versus to C. Ex. 1, salicylic acid being protonated was not strong enough to provide any antimicrobial preservation.

When comparing C. Ex. 4 to C. Ex. 3, the single addition of sodium bisulfite could help to significantly increase the antimicrobial preservation.

When comparing Ex. 2 to Ex. 1, the single addition of sodium bisulfite could help to significantly increase the antimicrobial preservation.

Hence, a preservative such as an alkali metal metabisulfite, an alkali metal sulfite, an alkali metal bisulfite, e.g. sodium bisulfite, an alkali metal hydrosulfite, or mixtures thereof can prevent any discoloration and enhance antimicrobial preservation of the personal cleansing composition at the recited pH from 5.5 to 7.

The following examples can illustrate the aspects of the present invention:

### Compositions (wt. %)

| Ingredients (wt. %) | Ex. 2 | Ex. 3 | Ex. 4 | Ex. 5 | Ex. 6 | Ex. 7 |
|---|---|---|---|---|---|---|
| Sodium cocoyl isethionate¹ | 2.00 | 2.00 | 2.00 | 2.00 | 2.00 | 2.00 |
| Sodium lauroyl sarcosinate² | 4.30 | 4.30 | 4.30 | 4.30 | 4.30 | 4.30 |
| Cocamidopropyl betaine³ | 7.75 | 7.75 | 7.75 | 7.75 | 7.75 | 7.75 |
| Cocamide MEA⁴ | 1.00 | 2.00 | 1.00 | 2.00 | 1.00 | 1.00 |
| PEG-120 methyl glucose | 0.12 | 1.00 | 0.12 | 1.00 | 0.12 | 0.12 |
| trioleate⁵ | | | | | | |
| PEG-120 methyl glucose | — | — | — | — | — | — |
| dioleate¹¹ | | | | | | |
| Ratio CMEA/PEG-120 | 8.3 | 2.0 | 8.3 | 2.0 | 8.3 | 8.3 |
| methyl glucose trioleate | | | | | | |
| Sodium salicylate⁶ | 0.40 | 0.40 | — | — | — | — |
| Sodium benzoate⁷ | 0.45 | 0.45 | — | — | — | — |
| Citric acid powder⁸ | q.s. | q.s. | q.s. | q.s. | q.s. | q.s. |
| | pH 6.1 | pH 6.1 | pH 6.1 | pH 6.1 | pH 6.1 | pH 6.1 |
| Sodium bisulfite⁹ | 0.10 | 0.10 | 0.10 | 0.10 | 0.10 | 0.10 |
| Fragrance | 1.0 | 1.0 | 1.0 | 1.0 | 1.0 | 1.0 |
| Sodium chloride¹⁰ | — | — | — | — | 1.0 | 2.0 |
| water | Balance | Balance | Balance | Balance | Balance | Balance |
| | to 100 | to 100 | to 100 | to 100 | to 100 | to 100 |
| Ingredients (wt. %) | Ex. 8 | Ex. 9 | Ex. 10 | Ex. 11 | Ex. 12 | Ex. 13 |
| Sodium cocoyl isethionate¹ | 2.00 | 2.00 | 2.00 | 2.00 | 2.00 | 2.00 |
| Sodium lauroyl sarcosinate² | 4.30 | 4.30 | 4.30 | 4.30 | 4.30 | 4.30 |
| Cocamidopropyl betaine³ | 7.75 | 7.75 | 7.75 | 7.75 | 7.75 | 7.75 |
| Cocamide MEA⁴ | 1.00 | 2.00 | 1.00 | 2.00 | 1.00 | 1.00 |
| PEG-120 methyl glucose | — | — | — | — | — | — |
| trioleate⁵ | | | | | | |
| PEG-120 methyl glucose | 0.12 | 1.00 | 0.12 | 1.00 | 0.12 | 0.12 |
| dioleate¹¹ | | | | | | |
| Ratio CMEA/PEG-120 | 8.3 | 2.0 | 8.3 | 2.0 | 8.3 | 8.3 |
| methyl glucose trioleate | | | | | | |
| Sodium salicylate⁶ | 0.40 | 0.40 | — | — | — | — |
| Sodium benzoate⁷ | 0.45 | 0.45 | — | — | — | — |
| Citric acid powder⁸ | q.s. | q.s. | q.s. | q.s. | q.s. | q.s. |
| | pH 6.1 | pH 6.1 | pH 6.1 | pH 6.1 | pH 6.1 | pH 6.1 |
| Sodium bisulfite⁹ | 0.10 | 0.10 | 0.10 | 0.10 | 0.10 | 0.10 |
| Fragrance | 1.0 | 1.0 | 1.0 | 1.0 | 1.0 | 1.0 |
| Sodium chloride¹⁰ | — | — | — | — | 1.0 | 2.0 |
| water | Balance | Balance | Balance | Balance | Balance | Balance |
| | to 100 | to 100 | to 100 | to 100 | to 100 | to 100 |

Definitions of Components
¹⁰Sodium chloride; Supplier Morton International
¹¹Glucamate ™ DOE-120; Supplier Lubrizol Advanced Materials

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

Every document cited herein, including any cross referenced or related patent or application, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A personal cleansing composition comprising:
a) a surfactant system, wherein the surfactant system comprises:
(i) from about 0.1% to about 5% of a fatty acyl isethionate surfactant by weight of the composition;
(ii) from about 0.5% to about 40% of a co-surfactant by weight of the composition;
wherein the composition is free of alkyl sulfate or alkyl ether sulfate type of surfactants;
b) from about 0.05% to about 5% of a of a non-ionic surfactant by weight of the composition, wherein the non-ionic surfactant comprises a fatty alkanolamide;
c) from about 0.01% to about 2.0% of a hydrophobically modified ethoxylated methyl glucoside by weight of the composition;
d) from about 0.01% to about 1.0% of a preservative by weight of the composition; and
e) wherein the pH is from about 5.5 to about 7.

2. The personal cleansing composition of claim 1, wherein the fatty acyl isethionate surfactant is an isethionate according to the general Formula (I): 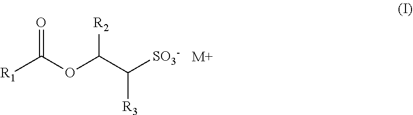
wherein R₁ is a saturated or unsaturated, straight or branched, alkyl or alkenyl chain with from 6 to 30 carbon atoms, R₂ and R₃ are each independently H or (C₁-C₄) alkyl, and M⁺ is an alkali metal; or M⁺ is an alkali-earth metal; or M⁺ is an ammonium or a substituted ammonium cation.

3. The personal cleansing composition of claim 1, wherein the fatty acyl isethionate surfactant is selected from the group consisting of sodium lauroyl isethionate, sodium lauroyl methyl isethionate, sodium oleoyl isethionate, sodium oleoyl methyl isethionate, sodium stearoyl isethionate, sodium stearoyl methyl isethionate, sodium myristoyl isethionate, sodium myristoyl methyl isethionate, sodium palmitoyl isethionate, sodium palmitoyl methyl isethionate, sodium cocoyl isethionate, sodium cocoyl methyl isethionate, a blend of stearic acid and sodium cocoyl isethionate, ammonium cocoyl isethionate, ammonium cocoyl methyl isethionate, and mixtures thereof.

4. The personal cleansing composition of claim 1, wherein the surfactant system further comprises:
from about 0.1% to about 10% of a fatty acyl sarcosinate surfactant by weight of the composition, according to the general formula (II): 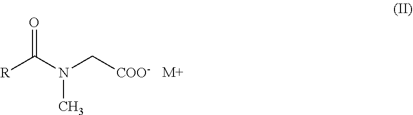
wherein R is a saturated or unsaturated, straight or branched or alkenyl and M+ is H, a sodium, potassium or ammonium cation.

5. The personal cleansing composition of claim 4, wherein the fatty acyl sarcosinate surfactant is chosen from sodium lauroyl sarcosinate, sodium cocoyl sarcosinate, sodium myristoyl sarcosinate, TEA-cocoyl sarcosinate, ammonium cocoyl sarcosinate, ammonium lauroyl sarcosinate, dimer dilinoleyl bis-lauroyl glutamate/lauroyl sarcosinate, lauroyl sarcosinate, isopropyl lauroyl sarcosinate, potassium cocoyl sarcosinate, potassium lauroyl sarcosinate, sodium oleoyl sarcosinate, sodium palmitoyl sarcosinate, TEA-lauroyl sarcosinate, TEA-oleoyl sarcosinate, TEA-palm kernel sarcosinate, or mixtures thereof.

6. The personal cleansing composition of claim 1, wherein the personal cleansing composition further comprises from about 0.05 to about 5% of an electrolyte by weight of the composition, wherein the electrolyte is chosen from sodium citrate, potassium citrate, calcium chloride, calcium bromide, zinc chloride, barium chloride, calcium nitrate, potassium chloride, sodium chloride, potassium iodide, sodium bromide, ammonium bromide, sodium sulfate, or mixtures thereof.

7. The personal cleansing composition of claim 1, wherein the personal cleansing composition further comprises from about 0.01 wt. % to about 2 wt. % of a fragrance component by weight of the composition.

8. The personal cleansing composition of claim 1, wherein the fatty alkanolamide is chosen from cocamide, cocamide methyl MEA, cocamide MEA, cocamide DEA, cocamide MIPA, lauramide MEA, lauramide DEA, lauramide MIPA, myristamide MEA, myristamide DEA, PEG-20 cocamide MEA, PEG-2 cocamide, PEG-3 cocamide, PEG-4 cocamide, PEG-5 cocamide, PEG-6 cocamide, PEG-7 cocamide, PEG-3 lauramide, PEG-5 lauramide, PEG-3 oleamide, PPG-2 cocamide, PPG-2 hydroxyethyl cocamide, PPG-2 hydroxyethyl isostearamide, soyamide DEA, oleamide MIPA, stearamide MEA, myristamide DEA, stearamide DEA, oleylamide DEA, tallowamide DEA, lauramide MIPA, tallowamide MEA, isostearamide DEA, isostearamide MEA, or mixtures thereof.

9. The personal cleansing composition of claim 1, wherein the fatty alkanolamide is chosen from cocamide methyl MEA, cocamide MEA, cocamide DEA, cocamide MIPA, lauramide MEA, lauramide DEA, lauramide MIPA, myristamide MEA, myristamide DEA, soyamide DEA, oleamide MIPA, stearamide MEA, stearamide DEA, oleylamide DEA, tallowamide DEA, lauramide MIPA, tallowamide MEA, isostearamide DEA, isostearamide MEA, or mixtures thereof.

10. The personal cleansing composition of claim 1, wherein the hydrophobically modified ethoxylated methyl glucoside is chosen from of PEG-120 methyl glucose dioleate, PEG-120 methyl glucose trioleate, PEG-20 methyl glucose sesquistearate, PEG-20 methyl glucose distearate, PEG-80 methyl glucose laurate, PEG-20 methyl glucose sesquilaurate, PEG-120 methyl glucose triisostearate, or mixtures thereof.

11. The personal cleansing composition of claim 1, wherein the weight ratio of the fatty alkanolamide to the hydrophobically modified ethoxylated methyl glucoside ranges from about 2 to about 12.

12. The personal cleansing composition of claim 1, wherein the fatty alkanolamide comprises cocamide MEA and the hydrophobically modified ethoxylated methyl glucoside is chosen from PEG-120 methyl glucose dioleate, PEG-120 methyl glucose trioleate, or mixtures thereof, wherein the weight ratio of the fatty alkanolamide to the hydrophobically modified ethoxylated methyl glucoside ranges from about 5 to about 12.

13. The personal cleansing composition of claim 1, wherein the preservative is chosen from an alkali metal metabisulfite, an alkali metal sulfite, an alkali metal bisulfite, an alkali metal hydrosulfite, or mixtures thereof.

14. The personal cleansing composition of claim 1, wherein the preservative is chosen from sodium metabisulfite, sodium sulfite, sodium hydrosulfite, potassium sulfite, sodium bisulfite, potassium bisulfite and mixtures thereof.

15. The personal cleansing composition of claim 1, wherein the co-surfactant comprises a zwitterionic surfactant comprising an alkyl betaine and/or an alkyl amidopropyl betaine.

16. The personal cleansing composition of claim 1, wherein the composition is free of direct dyes, oxidative dyes, parabens, or mixtures thereof.

17. A method of increasing the stability of a personal cleansing composition in terms of phase, color, and microbial stability comprising the step of forming a personal cleansing composition of claim 1.
